(19) United States
(12) Patent Application Publication
(10) Pub. No.: US 2018/0064502 A1
(43) Pub. Date: Mar. 8, 2018

(54) TUBE CLIP ORGANIZER

(71) Applicant: Biometrix Ltd., Jerusalem (IL)

(72) Inventor: Matan SHAMIR, Jerusalem (IL)

(21) Appl. No.: 15/683,945

(22) Filed: Aug. 23, 2017

Related U.S. Application Data
(60) Provisional application No. 62/384,283, filed on Sep. 7, 2016.

(51) Int. Cl. A61B 50/24; F16B 2/10; F16M 13/02; F16B 2/00; F16B 2/22

(52) U.S. Cl. CPC A61B 50/24; F16B 2/10; F16B 2/22; F16B 2/005; F16M 13/022

(57) ABSTRACT

An organizer comprising: a first arm; a second arm; one or more port, each port disposed on an arm, each port configured for attachment thereto of a medical device portion; a first grip on the first arm comprising a first concavity; a second grip on the second arm comprising a second concavity; a hinge connecting the arms, where arms extend distally from the hinge and are rotatable about the hinge; and a ratchet-lock, which locks the arms at plurality of distances, each distance separated from adjacent distances by a minimum step size; wherein the arms are flexible enough in a direction perpendicular to an axis of the hinge such that total elastic deformation of the arms is at least a quarter of the minimum step size under a level of force easily applied manually.

### RELATED APPLICATIONS

This application claims the benefit of priority under 35 USC §119(e) of U.S. Provisional Patent Application No. 62/384,283, filed on Sep. 7, 2016, the contents of which are incorporated herein by reference in their entirety.

### FIELD AND BACKGROUND OF THE INVENTION

The present invention, in some embodiments thereof, relates to a device which clips onto a support and, more particularly, but not exclusively, to an organizer device which clips onto a support where the organizer includes a plurality of ports for attachment thereto of medical device portions.

U.S. Design patent D678533 S1 discloses “an ornamental design for an arterial blood pressure transducer module clip”.

US Patent Application No. 2014/0194778 discloses “An in-line sampling syringe for selectively introducing and aspirating a fluid from a downstream fluid conduit connected to a catheterized patient. The syringe includes a housing with a distal component defining a distal chamber having a proximal opening and a distal outlet port to be coupled to a downstream fluid conduit; and a pair of proximal wall components assembled together in an airtight manner defining a proximal chamber having a proximal opening for coupling to an external source of fluid and a distal opening that is coupled to the proximal opening of the distal component. The syringe also includes an elongated volume regulator positioned inside the housing. A curved element is positioned at and in alignment with the distal outlet and retention elements are defined in the distal component at the distal outlet port to mount and retain the curved element at the distal outlet port while enabling flow around the curved element relative to the distal outlet port. A conduit extends from the proximal opening of the proximal chamber through the elongated volume regulator to the curved element at the distal outlet port . . . A clip (771) for mounting syringe (700) on a mounting board (not shown) is removably attached to fluid chamber (710) via grooves (711) situated on opposing sides thereof.”

### SUMMARY OF THE INVENTION

According to an aspect of some embodiments of the present invention there is provided an organizer, configured to be mounted onto rod supports of different cross sectional sizes, where the organizer provides a hub for attachment thereto of a plurality of medical device portions, the organizer comprising:

a first arm;

a second arm;

one or more port, each port disposed on an arm, each port configured for attachment thereto of a medical device portion;

a first grip disposed on the first arm comprising a first concavity;

a second grip disposed on the second arm comprising a second concavity;

a hinge connecting the arms, where the first arm and the second arm extend distally from the hinge and are rotatable about the hinge; and

a ratchet-lock, which, when locked, connects the arms, the ratchet lock comprising:
a first ratchet-lock portion connected to the first arm;
a second ratchet-lock portion connected to the second arm;

wherein the arms are lockable by the ratchet-lock at a plurality of distances, each distance separated from adjacent distances by at least a minimum step size;

wherein, to close the organizer onto a rod support, the arms are rotated about the hinge towards each other bringing the concavities of the grips to face each other and into at least partial contact with the rod support;

wherein one or both of the arms are flexible enough in a direction perpendicular to an axis of the hinge such that total elastic deformation of the arms is at least a quarter of the minimum step size under a level of force easily applied manually.

According to some embodiments of the invention, the arms rotate about the hinge, moving in a plane perpendicular to an axis of the hinge;

wherein the first concavity comprises a first portion;

wherein the second concavity comprises a second portion;

wherein the first portion and the second portion occupy a same location perpendicular to the plane and different first and second locations in the plane when the arms are secured on rod supports with a cross sectional dimension less than a first support rod dimension.

According to some embodiments of the invention, the first concavity comprises a third portion;

wherein the third portion occupies the same location perpendicular to the plane and occupies a third region, in the plane, different to the first and second region.

According to some embodiments of the invention, the third portion occupies a space between the first concavity and the second concavity perpendicular to the plane.

According to some embodiments of the invention, the first concavity includes two first portions and the second concavity includes two second portions, the first portions located at different edges of the first concavity and the second portions located at different edges of the second concavity.

According to some embodiments of the invention, the first portion at an edge of the first grip and the second portion is at an edge of the second grip.

According to some embodiments of the invention, the first support rod dimension is a sum of the depths of the first and the second concavity, a depth of a concavity measured as the largest distance perpendicular to an axis of the hinge and perpendicular to a tangential plane connecting two distal edges of the concavity.

According to some embodiments of the invention, the first ratchet-lock portion is connected to a distal end of the first arm.

According to some embodiments of the invention, the second ratchet-lock portion is connected to a distal end of the second arm.

According to some embodiments of the invention, the first concavity comprises a first elastically compressible grip interface.

According to some embodiments of the invention, the second concavity comprises a second elastically compressible grip interface.

According to some embodiments of the invention, one or both of the arms are flexible enough in a direction perpendicular to an axis of the hinge such that total elastic deformation of the arms is at least the minimum step size under a level of force easily applied manually. According to some embodiments of the invention, the force easily applied manually is about 30 N.

According to some embodiments of the invention, one or both of the arms are flexible enough in a direction perpendicular to the axis of the hinge to such that total elastic deformation of the arms is at least a quarter of the step size under a force of less than 100 N.

According to some embodiments of the invention, the total deformation of the arms, by 1-3 mm provides an elastic force of at least 2 N.

According to some embodiments of the invention, the total deformation is defined as a sum of a first arm deformation and a second arm deformation where deformation of an arm is a maximum deviation of the arm measured from a relaxed arm shape to the deformed arm shape in a plane of rotation of the arms.

According to some embodiments of the invention, the first concavity and the second concavity are in a plane perpendicular to an axis of the hinge.

According to some embodiments of the invention, the first grip, perpendicular to the hinge axis, includes a first and a second distal end, the ends orientated between 30-160° from each other.

According to some embodiments of the invention, the second grip, perpendicular to the hinge axis, includes a first and a second distal end, the ends orientated between 30-160° from each other. According to some embodiments of the invention, the ends of the first grip and the ends of the second grip are orientated between 70-110° from each other.

According to some embodiments of the invention, a trough of the first grip has a radius of curvature of about half of a minimum distance between the first grip and the second grip.

According to some embodiments of the invention, a trough of the second grip has a radius of curvature of about half of a minimum distance between the first grip and the second grip.

According to some embodiments of the invention, the minimum distance is defined by a final ratchet-lock locking position, where the ratchet is locked into a position where the arms are minimally separated from each other.

According to some embodiments of the invention, the first ratchet-lock portion comprises a plurality of teeth, where adjacent teeth are separated by the minimum step size, the plurality of teeth comprising:

a first tooth located most distant from the first arm;

a final tooth located closest to the first arm; and

wherein the second ratchet-lock portion comprises a pawl;

wherein the minimum distance between the first grip and the second grip is when the pawl is locked against the final tooth.

According to some embodiments of the invention, the minimum distance is defined by a shape of the first grip and a shape of the second grip.

According to some embodiments of the invention, a distance between each the grip and the hinge is at least 2 times a diameter of a support associated with a largest ratchet-lock locking position.

According to some embodiments of the invention, a port on the first arm comprises a slide attachment socket.

According to some embodiments of the invention, the port of the first arm comprises a snap fit lock.

According to some embodiments of the invention, the slide attachment socket comprises an inlet surrounded by a framework overhanging the inlet.

According to some embodiments of the invention, the first arm comprises three ports, each port comprising a slide attachment socket, each socket comprising an inlet surrounded by a framework overhanging the inlet.

According to some embodiments of the invention, a port of the second arm includes a snap-fit attachment socket.

According to some embodiments of the invention, the snap-fit attachment socket includes at least one arm which is elastically deflected by insertion of a portion of a medical device into the socket, the at least one arm holding the portion of a medical device in the socket.

According to some embodiments of the invention, the ratchet-lock comprises:

a plurality of teeth attached to the first arm, where adjacent teeth are separated by the minimum step size; and

a pawl attached to the second arm.

According to some embodiments of the invention, the arms are composed of plastic.

According to some embodiments of the invention, the first concavity and the second concavity have a high friction outer surface.

According to some embodiments of the invention, the grip interfaces have a high friction outer surface.

According to some embodiments of the invention, the grip interfaces are composed of silicone rubber.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention.

In this regard, the description taken with the drawings makes apparent to those skilled in the art how embodiments of the invention may be practiced.

In the drawings:

FIG. 1A is a simplified schematic isometric view of an exemplary organizer secured to a support, according to some embodiments of the invention;

FIG. 1B is a simplified schematic isometric view of an exemplary organizer secured to a support, according to some embodiments of the invention;

FIG. 2 is a simplified schematic side view of an organizer, according to some embodiments of the invention;

FIG. 3 is a flow chart of a method of use of an organizer, according to some embodiments of the invention;

FIG. 4A is a simplified schematic top view of an organizer, where a connector is in an open position, according to some embodiments of the invention;

FIG. 4B is a simplified schematic top view of the organizer of FIG. 4A, tightened onto a support, according to some embodiments of the invention;

FIG. 5 is a simplified schematic top view of an organizer tightened onto a support, according to some embodiments of the invention;

FIG. 6A is a simplified schematic top view of an exemplary organizer secured onto a maximum diameter pole support, according to some embodiments of the invention;

FIG. 6B is a simplified schematic top view of the organizer of FIG. 6A, where the organizer is configured to fit a minimum pole support, according to some embodiments of the invention;

FIG. 7 is a simplified schematic top view of an organizer, according to some embodiments of the invention;

FIG. 8 is a simplified schematic top view of an organizer, according to some embodiments of the invention.

FIG. 9A is a simplified schematic isometric view of an organizer including grips shaped to interleave, according to some embodiments of the invention;

FIG. 9B are simplified schematic plan views of the grip interfaces of FIG. 9A, according to some embodiments of the invention;

FIG. 9C, FIG. 9D and FIG. 9E are simplified schematic cross sections of an organizer, the cross section taken perpendicular to an axis of the hinge, according to some embodiments of the invention;

FIG. 10 illustrates interleaving grips on a support, according to some embodiments of the invention;

FIG. 11 illustrates overlapping grips, on a support, according to some embodiments of the invention;

FIG. 12 is a simplified schematic top view of an organizer, according to some embodiments of the invention;

FIG. 13 is a simplified schematic top view of an organizer device including more than two grips, according to some embodiments of the invention;

FIG. 14 is a simplified schematic isometric view of a portion of an exemplary organizer, according to some embodiments of the invention; and

FIG. 15A is a simplified schematic isometric view of a port plate, according to some embodiments of the invention; and

FIG. 15B is a simplified schematic isometric view of an attachment clamp, according to some embodiments of the invention.

### DESCRIPTION OF SPECIFIC EMBODIMENTS OF THE INVENTION

The present invention, in some embodiments thereof, relates to a device which clips onto a support and, more particularly, but not exclusively, to an organizer device which clips onto a support where the organizer includes a plurality of ports for attachment thereto of medical device portions.

### Overview

A broad aspect of some embodiments of the invention relates to an organizer device which provides a hub for attachment thereto of a plurality of medical devices, where the organizer securely attaches to a range of different sized rod supports while being easy to remove. In an exemplary embodiment, the support is an IV pole.

In an exemplary embodiment, the organizer includes two arms connected at their proximal ends by a hinge and a ratchet mechanism to lock distal ends of the arms together. In some embodiments, a support is held within a space defined between the two arms and the ratchet mechanism.

In some embodiments, elastic force provided by an elastic deformation of one or more portion of the organizer provides increases friction between the organizer and the support, holding the organizer onto the support. In some embodiments, one or more arm deforms elastically, for example, bending around the support (e.g. when a user locks the arms together).

In some embodiments, the elastically deformable portion/s (e.g. arm/s) are sufficiently resistant to deformation (e.g. rigid and/or or of high stiffness) that a small deformation generates enough elastic force to hold the organizer onto the support. In some embodiments, locking the organizer (e.g. locking the arms together using the ratchet lock) maintains the deformation of the portion/s e.g. in some embodiments, holding the organizer onto the support.

In some embodiments, the organizer is easily attached and removed. For example, the organizer arms freely move about the hinge until the organizer is locked onto the support. For example, in some embodiments, it is not necessary to overcome elastic forces when positioning the organizer on a support and/or the support within the organizer. For example, in some embodiments, a small amount of force is overcome positioning the arms on a rod, e.g. an amount of elastic force which is easily overcome manually by a user e.g. using one hand. For example, in some embodiments, when the mechanism locking the arms is unlocked, the arms freely rotate about the hinge, potentially enabling removal and/or repositioning of the organizer.

In some embodiments, the organizer includes one or more port on each arm. Ports facilitate connection of medical devices to the organizer. In some embodiments each port includes one or more protrusion and/or one or more inlet and a medical device includes a mating topography (and/or a medical device is attached to an intermediate element which includes a mating topography). Exemplary types of port include slide attachment where, for example, a medical device is attached by sliding into an inlet e.g. from above, snap-fit attachment.

In some embodiments, one or more slide attachment port includes one or more locking element (e.g. a stopper) which prevents the medical device (and/or intermediate element) from sliding out of the port once inserted.

In some embodiments, the lock (e.g. ratchet lock) prevents the arms from opening, in some embodiments, without preventing the arms from being pushed closer together.

In some embodiments, the organizer includes one or more grip disposed on one or more arm. In some embodiments, an organizer includes two grips, one on each arm, where the grips are positioned on the arm/s such that they contact a support (e.g. facing each other as the arms are brought towards each other). In some embodiments, the organizer includes a grip element on each arm where the grip elements are located on different rotational sides about the hinge of the arms, the grips thereby coming into contact as the sides of the arms on which the grips are located are brought together. In some embodiments, the grips are positioned on the arms at the same distance from the hinge.

In some embodiments, the grip elements are positioned on the arms to contact (e.g. centrally) a range of different sized supports. For example, in some embodiments, a grip is positioned on an arm (and/or two grips, one on each arm are each positioned) at a distance distal of the hinge which is least 2 times a diameter of a maximum diameter support, where, in some embodiments, a maximum diameter support is the support which fits between the arms and/or between a grip and an arm and/or between two grips when the ratchet lock is secured at the largest ratchet lock step.

In some embodiments, additionally or alternatively to elastic deformation of arm/s, one or more grip includes a portion which elastically deforms when a user locks the arms together, e.g. to at least partially provide the elastic force which holds the organizer onto the support and/or to increase friction between the support and the grip. In some embodiments, compression of a grip increases contact area between the grip and the support. In some embodiments, one or more grip includes a non-compressible and/or rigid grip base on which one or more compressible and/or high friction grip interface are disposed.

In some embodiments, the organizer includes one or more grip sized and/or shaped to maximize contact between the organizer and the support, for a range of different sized supports.

Potentially, increased contact area between the organizer and a support prevents movement of the organizer on the support e.g. due to friction between the organizer and the support.

In some embodiments, one or more grip includes a concave portion, for example, orientated such that the grip receives a support with a concave surface. In some embodiments, one or more grip is concave in a plane perpendicular to an axis of the hinge.

In some embodiments, the organizer attaches securely to a range of different sized supports. For example, in some embodiments, the ratchet lock connecting the arms has a range of locking positions which enable the organizer to be secured to different sized supports held between the organizer arms. In some embodiments, a maximum size of support to which the organizer can be securely attached is defined by the largest ratchet lock position. For example, in some embodiments, a minimum size of support to which the organizer can be securely attached is defined by a minimum separation between the arms e.g. defined by the size of the hinge and/or shape of the arms and/or size and/or shape of grips.

An aspect of some embodiments of the invention relates to an organizer where two grips, one on disposed on each arm are sized and/or shaped and/or positioned on the organizer such that the extent to which the grips protrude into a space between the arms does not define a minimum size of support to which the organizer is sized.

For example, in some embodiments, two grips, one disposed on each arm have portions which overlap in space in a direction between the arms, as the arms are brought towards each other (e.g. for supports with cross section under a certain dimension). In some embodiments, a first grip and a second grip each have a portion where the portions overlap (e.g. occupy a same space) in a plane connecting the two arms. In some embodiments, two grips, a first grip disposed on a first arm and a second grip disposed on a second arm have portions which overlap parallel to an axis of the hinge, for example, the first grip having a portion which occupies the same space perpendicular to an axis of the hinge and a different space parallel to an axis of the hinge.

For example, in some embodiments, the organizer includes grips which interleave as the arms are brought towards each other. In an exemplary embodiment, two grips, one on disposed on each arm include one or more matching protrusions and inlet (e.g. the protrusion on one grip arm and the inlet on another grip), a protrusion moving into an inlet as the arms (and grips) are brought towards each other. Potentially, interleaving and/or overlapping grips afford attachment of the organizer to an increased range of support pole diameters.

In an exemplary embodiment one or more grip has a shape in a direction in which the grip receives a support and/or a direction between the arms (e.g. a direction perpendicular to an axis of the hinge) where the shape is a v-shape with a rounded trough. In some embodiments, the organizer includes two facing grips, each including a v-shape cross section where the grips overlap and/or interleave. In some embodiments, the overlapping and/or interleaving is sufficient such that the smallest diameter support corresponds to a curvature of the rounded trough of the grip shape.

In some embodiments, the organizer arms are not straight, for example, in some embodiments, a middle section of one or more organizer arm curving away from a straight line connecting the proximal and distal ends of the arm. The direction of the curvature of the arm is such that the space between the arms is increased.

Potentially, this enables use of the organizer with larger supports for a given ratchet size.

In some embodiments, a distance between the hinge and the ratchet-lock is long, for example, with respect to a range of size supports (e.g. as defined as the range between a minimum sized support and a maximum sized support as defined elsewhere in this document) to which the organizer is attachable. For example, in an exemplary embodiment, a distance between a hinge and a ratchet mechanism is about 14 cm,a minimum size support is 14 mm diameter, and a maximum sized support is 40 mm diameter, the length of the organizer being about 5 times a range of sizes of support. In some embodiments, the length of the organizer is 3-10 times, or higher, or lower, or intermediate multiples or ranges of a range of sizes of support.

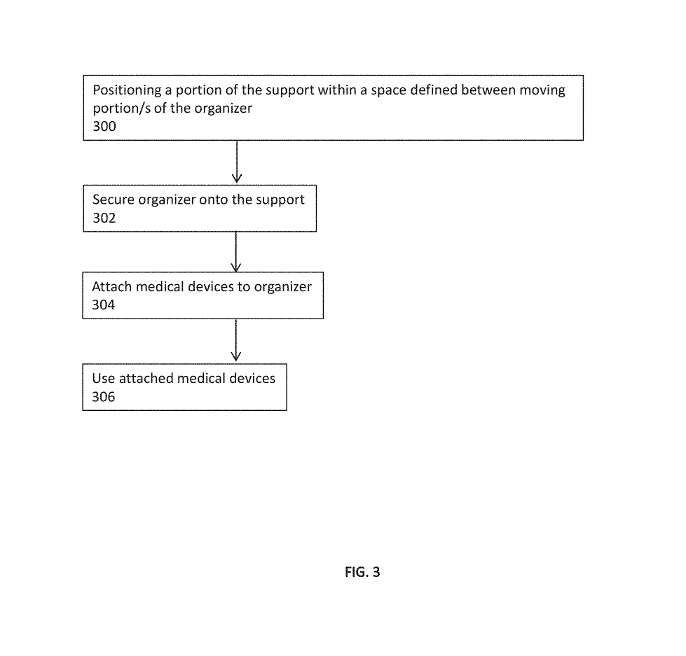

In some embodiments, a distance between the grips and the ratchet-lock is long, for example, with respect to a range of size supports (e.g. as defined as the range between a minimum sized support and a maximum sized support as defined elsewhere in this document) to which the organizer is attachable. For example, about 2 times, or 1-3 times, or higher, or lower, or intermediate multiples or ranges of a range of sizes of support. This measurement is relevant, for example, in embodiments, where the organizer arms mainly and/or only bend distally of the grips, e.g. as illustrated in FIG. 4B.

Potentially, a large separation between the ratchet-lock and the support and/or grips and/or hinge improves spatial resolution of the device, e.g. potentially enabling attachment of the device to a more continuous range of size of support, for a given ratchet step size.

Potentially, increasing separation between the ratchet-lock and the support and/or grips increases a force applied by the ratchet onto the support through the arms and grips.

Potentially, increasing separation between the ratchet-lock and the support and/or grips means that the force a user has to apply in order to secure the organizer onto a support (e.g. sufficient force to conform grip interfaces to the outer topography of the support) is reduced, when the user is applying force at the ratchet, and/or between the ratchet and the support.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

### Exemplary Organizer

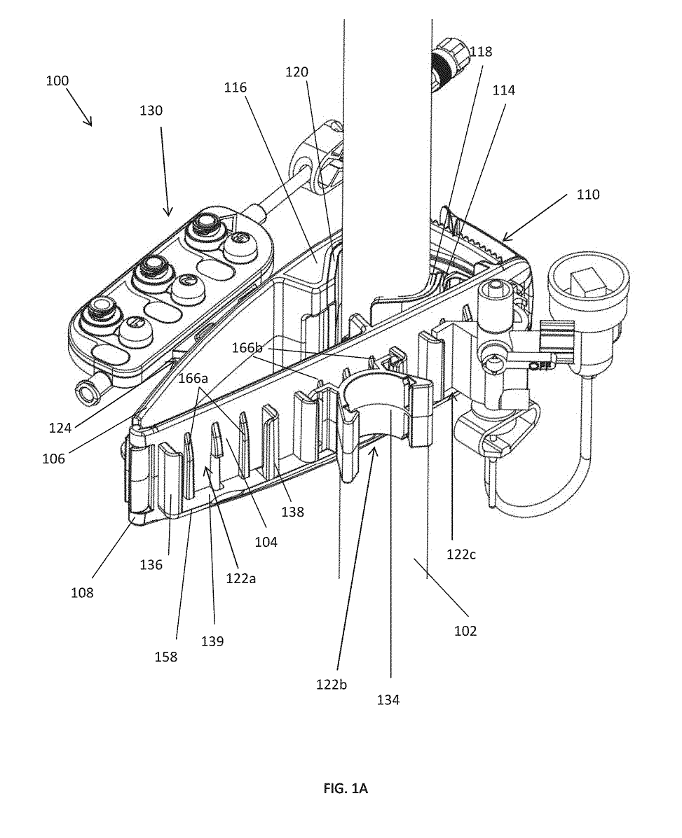

FIG. 1A is a simplified schematic isometric view of an exemplary organizer 100 secured to a support 102, according to some embodiments of the invention. FIG. 1B is a simplified schematic isometric view of an exemplary organizer 100 secured to a support 102, according to some embodiments of the invention. In some embodiments, FIG. 1A and FIG. 1B show different views of the same organizer device.

In some embodiments, the organizer 100 includes a first arm 104 and a second arm 106.

In some embodiments, arms 104, 106 are connected by a hinge 108 at proximal ends of the arms. In some embodiments, arms 104, 106 rotate about hinge 108, e.g. to enlarge and/or reduce spaces between the arms.

In some embodiments, hinge 108 is formed by interlocking portions of arms 104, 106. In some embodiments, hinge 108 is formed by interlocking portion of arms and a pin e.g. hinge 108 is a barrel hinge. In some embodiments, hinge 108 is a living hinge.

In some embodiments, arms 104, 106 are lockable by a connector 110, where connector 110 connects the arms distally from hinge 108. In some embodiments, connector 110 connects distal ends of arms 104, 106. In some embodiments, arms 104, 106 extend distally past connection between the arms by connector 110.

In some embodiments, connector 110 is an adjustable connector which connects the arms at different separations. In an exemplary embodiment connector 110 is a ratchet lock mechanism. Alternative connector mechanisms include clamp, tie.

In some embodiments, ratchet lock 110 has steps with uniform step sizes. In an exemplary embodiment, ratchet lock 110 has 20 uniformly sized steps (e.g.

uniformly sized teeth). Alternatively or additionally, in some embodiments, ratchet lock 110 has different sized teeth.

In an exemplary embodiment, teeth 128 are small in comparison to the minimum size support for which the organizer is suitable, for example, where a step size between teeth is at least double, or at least 5 times, or at least 10 times, or 2-50 times, or lower or higher or intermediate multiples or ranges, a minimum support diameter (minimum support diameter e.g. defined by and/or equal to a minimum space perpendicular to an axis of hinge 108, between grips 114, 116).

In some embodiments, teeth 128 are small in comparison to a range of support sizes. For example, where a ratchet lock step size (e.g. tooth size) is less than a quarter, or less than a tenth, or less than a twentieth, or less than a hundredth, or 0.1-25%, of 1-10% of a size range between a minimum and a maximum sized support (minimum and maximum sized supports e.g. as defined elsewhere in this document).

A potential advantage of small teeth is adjustability of the device e.g. ability of the device to be used with many different sized supports (e.g. a continuous range of support sizes).

In some embodiments, ratchet lock 110 has a teeth sized and/or positioned for specific support sizes, for example, including a discrete number of teeth (teeth optionally of different size), each tooth corresponding to a support size.

In some embodiments, arms 104, 106 freely rotate about hinge 108 when the arms are not connected by connector 110. In some embodiments, arms 104, 106 are rotatable to a limited range of angles e.g. in some embodiments, a shape of the arm/s and or hinge and/or protrusion/s on the arm/s (e.g. rail 136, FIG. 1A) prevent opening of the arms beyond a maximum separation.

In an exemplary embodiment, e.g. as illustrated in FIG. 1A and FIG. 1B, arms 104, 106 have a shape, when the arms are elastically relaxed, where each arm bends away from a straight line connecting the proximal and the distal ends of the arm (the line also e.g. perpendicular to an axis of hinge 108). In some embodiments, the direction of bending of the arm/s is away from the space between the arms in which the pole support is located (e.g. each arm bends away from the other arm). Potentially, this bending of the arms enables a shorter ratchet mechanism, given a range of poles.

In some embodiments, e.g. as illustrated in FIG. 1A and FIG. 1B the bent shape is a gently curved shape. Alternatively, in some embodiments, one or both arms has a different bent shape, e.g. a v-shape. Alternatively, in some embodiments, one or both of the arms is straight.

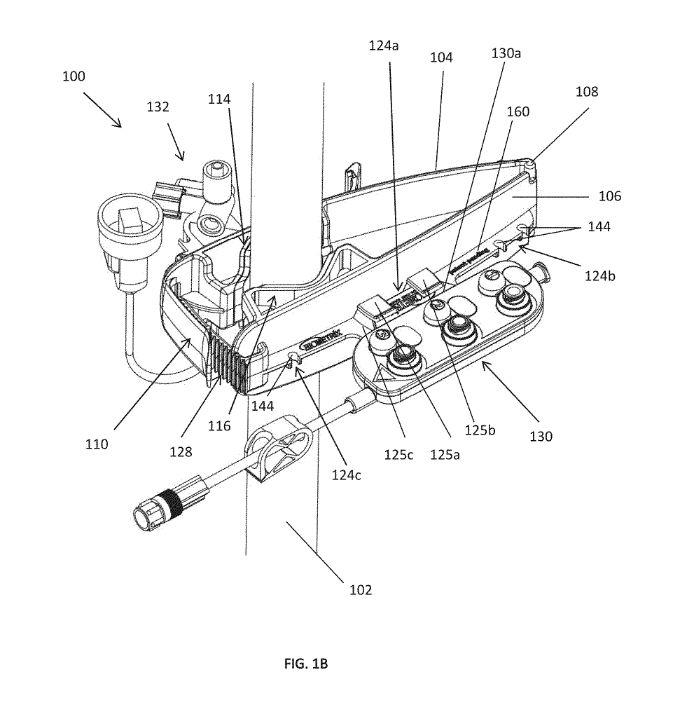

In some embodiments, one or more arm includes a shape where the arm is larger in a dimension parallel to an axis of the hinge at some portions of the arm. FIG. 2 is a simplified schematic side view of an organizer 200, according to some embodiments of the invention. In some embodiments, one or more arm e.g. first arm 204, has a dimension parallel to an axis of hinge 208 which is larger in a central portion of the arm between hinge 208 and a ratchet lock 210. For example, in an exemplary embodiment, a height of the organizer (dimension of the organizer parallel of an axis of hinge 208) is about 40 mm (e.g. height as illustrated on FIG. 2) compared with a height of the organizer at the hinge of about 33 mm (e.g. height as illustrated on FIG. 2). In some embodiments, a height of an arm at a midpoint of the arm between the hinge and the ratchet is 1.1-1.5 times, or about 1.2 times, the height of the arm at the hinge and/or ratchet. In an exemplary embodiment, an organizer arm gradually increases in height from a hinge distally towards a midpoint of the arm between the hinge and the ratchet lock. In an exemplary embodiment, an organizer arm gradually increases in height from a ratchet lock proximally towards a mid-portion of the arm between the hinge and the ratchet lock.

In some embodiments, organizer 100 includes one or more grip disposed on arm/s where one or more grip is shaped and/or positioned on arm/s to maximize contact with a support. In some embodiments, one or more grip protrudes into a space between arms 104, 106. Alternatively, in some embodiments, one or more grip is recessed within an arm.

In an exemplary embodiment, one or more grip includes a grip base, for example 116, 114 on which a grip interface is disposed, for example grip interfaces 118, 120. In some embodiments, grip bases are, for example, formed using a rigid material (e.g. plastic) onto which a grip interface comprising compressible and/or high friction material is disposed.

In an exemplary embodiment, grip interfaces 118, 120 include rubber e.g. silicone rubber. In some embodiments, grip interfaces 118, 120 include sponge, e.g. sponge rubber.

In some embodiments, organizer 100 includes a plurality of ports 122a, 122b, 122c, 124 for attachment of medical devices to the organizer.

In some embodiments, a length of the organizer, e.g. as measured from the hinge to the ratchet mechanism is 10-20 cm, or 12-17 cm, or 13-16 cm, or about 15 cm.

### Exemplary Method of Use

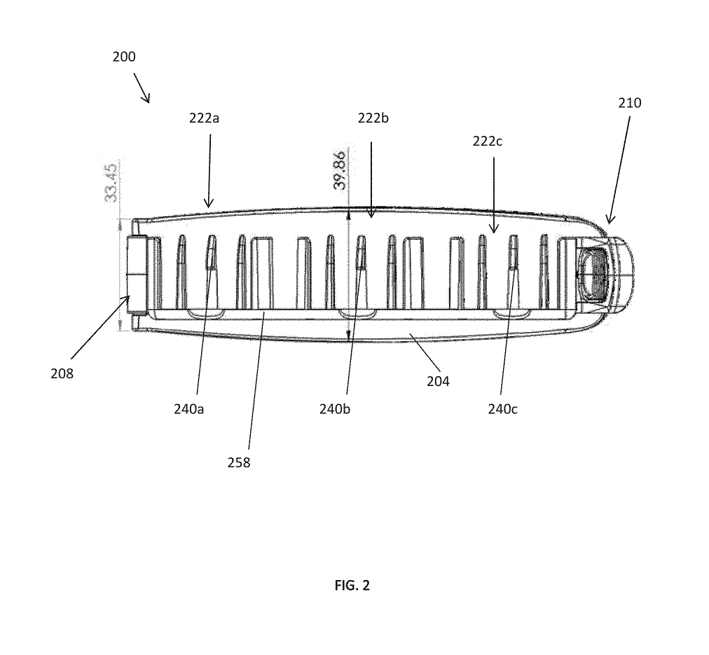

FIG. 3 is a flow chart of a method of use of an organizer, according to some embodiments of the invention.

At 300, the organizer is positioned at a desired position on the support, for example, at a desired height on a support rod (e.g. support 102, support 602) with the support rod positioned within a space between moving portion/s of the organizer. The organizer is then closed around the support. Closing of the organizer, for example, is by rotating portion/s (e.g. arm/s 104, 106,) about a hinge (e.g. hinge 108).

At 302, the organizer is secured, for example, by a user applying a force (e.g. manually) to the organizer, elastically deforming at least one portion of the organizer.

Where elastic deformation, is, for example, of one or more arm (e.g. first arm 106 and/or second arm 108) and/or of one or more grip interface (e.g. first grip interface 118 and/or second grip interface 120. Where, for example, elastic deformation of the arm/s is by bending and/or elastic deformation of grip interface/s is by compression.

In an exemplary embodiment, a user applies force by moving (e.g. grasping and/or pinching) distal ends of the two arms (e.g. arms 104, 106) towards each other.

In some embodiments, the ratchet lock (e.g. 110) locks at successive pawl-tooth positions as the arms are moved towards each other. Alternatively, in some embodiments, the user manually locks the ratchet device, e.g. by inserting a pawl into a tooth position while applying pressure to the arms.

In some embodiments, a level of force required to close and/or lock the organizer onto a support is a level of force which is easily applied manually by a user.

For example, a force easily applied using one hand. For example, a level of force as described elsewhere in this document (e.g. about 30 N).

In some embodiments, a user secures the organizer to an IV pole at a vertical level which is the same level as the patient's heart. In some embodiments, the organizer is secured onto a vertically orientated IV pole long axes of the arms, for example, orientated horizontally e.g. as illustrated in FIG. 1A. Alternatively, in some embodiments, the organizer is secured to a rod support with a different orientation and the arms, in some embodiments, have a corresponding different orientation. For example, in some embodiments, the rod support is a bed rail orientated horizontally and, in some embodiments, the organizer is attached to the bed rail with long axes of the arms running vertically.

At 304, a user attaches medical device/s to the organizer (e.g. portion/s of each medical device are attached to the organizer). In some embodiments, the organizer provides a hub for portions of medical devices attached to and/or in close proximity to a patient. Exemplary medical devices include; syringe (where, e.g. an intermediate element attached to the organizer is a syringe hanger and/or a syringe holder), manifold, pressure transmitter. Alternatively or additionally, in some embodiments, a user attaches one or more medical device to the organizer before the organizer is secured onto the support (e.g. step 304 is performed before and/or after step 302).

At 306, medical devices are used, e.g. connected to the patient. Alternatively, in some embodiments, a medical device is attached to the patient and then connected to the organizer.

### Exemplary Elastic Deformation

FIG. 4A is a simplified schematic top view of an organizer 400, where a connector 410 is in an open position, according to some embodiments of the invention. FIG. 4B is a simplified schematic top view of the organizer of FIG. 4A, tightened onto a support 402, according to some embodiments of the invention. FIG. 4B illustrates the organizer after tightening of the organizer onto a support 402.

In FIG. 4A and FIG. 4B a cross section of a support 402 is illustrated.

FIG. 4B illustrates elastic deformation of arms 404, 406 of organizer 400. A portion of arms 404, 406 (e.g. a portion of the arm/s proximal to a connecting mechanism 410) are bent away from a relaxed position (illustrated as dashed lines in FIG. 4B and illustrated in FIG. 4A).

In some embodiments, when the organizer is locked onto the support, the arm/s are bent a small amount (e.g. each point along a surface of one or more arm bending by at most 0.1-5, or 0.5-5 mm or 0.5-3 mm, or lower or higher or intermediate ranges or values, from a relaxed arm position).

In some embodiments, the arms are sufficiently flexible such that they deform by at least two ratchet lock step sizes, at least a ratchet lock step size, or at least half a ratchet lock step size, or at least quarter of ratchet lock step size, or at least an eighth of a ratchet lock step size, or at least a tenth of a ratchet lock step size, under reasonable and/or easily applied manual pressure applied by a user. Where, for, example, reasonable manual pressure applied by a user is less than 100 N, or less than 70 N, or less than 50 N, or about 30 N.

In an exemplary embodiment, the force on the arms required to secure the organizer is about 30 N.

In some embodiments, depending on where a user applies force, different levels of force are required to secure the organizer. For example, if the user applies force to a more rigid portion of the arms, for example, around the grips (rigidity and/or propensity of different portions of the arms to bend as described, for example, elsewhere in this document), a higher level of force is required to deform the arms sufficiently to secure the organizer.

For example, as described elsewhere in this document (e.g. with respect to FIG. 1A and FIG. 1B), in an exemplary embodiment, ratchet lock 110 includes 20 teeth with uniform spacing between the teeth. In some embodiments, bending of the arms (e.g. by 0.1-2 ratchet step sizes) corresponds with bending of the arms by 0.5-10% of the ratchet length.

In some embodiments, the portion/s which elastically deform are sufficiently resistant to deformation (e.g. rigid) such that deformation of the portion/s when locking the organizer onto the support provides sufficient force to increase friction between the organizer and the support (e.g. between the grip/s and the support) to hold the organizer onto the support. For example, in some embodiments, friction between the organizer and the grips is more than weight of the medical device portion/s attached to the support and/or tensile force of medical device portion/s on the organizer and/or weight of the organizer itself.

In some embodiments, deformation of the arms by a ratchet lock step size, or half a ratchet lock step size, or quarter of ratchet lock step size, or an eighth of a ratchet lock step size, or a tenth of a ratchet lock step size is sufficient to securely hold the organizer onto a support, for example, of at least 2 N, or at least IN or of 0.5-20 N, or 1-10 N or lower or higher or intermediate ranges or forces.

In some embodiments, the arms are sufficiently rigid that elastic deformation of the arms by 0.1-5 mm or 0.1-3 mm or 0.1-1 mm or lower or higher or intermediate ranges or deformations provides an elastic force sufficient to securely hold the organizer onto a support, for example, of at least 2 N, or at least IN or of 0.5-20 N, or 1-10 N or lower or higher or intermediate ranges or forces.

In some embodiments, both arms 404, 406 elastically deform. In some embodiments, only one arm (e.g. first arm 404 or second arm 406) elastically deforms. In some embodiments, first arm 404 and second arm 406 bend equally. Alternatively, in some embodiments, one arm bends more than the other, for example, as illustrated in FIG. 4B where second arm 406 bends more than first arm 404.

In some embodiments, different portions of an arm bend by different extents.

For example, in some embodiments, the arms bend less around the grips 114, 116 e.g. associated with reinforcing of the arms by the grip bases.

For example, as illustrated by FIG. 4B where arms 404, 406 are illustrated as only bending distally of grips 414, 416. In some embodiments, the organizer is designed such that bending of the arms is concentrated at certain portions of the arms. For example, in some embodiments, a portion of the arm associated with a port, is reinforced (e.g. reinforcement along the arm perpendicular to an axis of the hinge at the area of the port). Potentially, reinforcement at an area of a port reduces bending of the arm at the area of the arm around the port e.g. potentially preventing deformation of the port and/or movement of the medical device when attaching and/or removing the organizer from the support and/or the medical device from the port.

Referring now back to FIG. 1A and FIG. 1B, in some embodiments first arm 104 includes a beam 158 and/or second arm 106 includes a beam 160. In some embodiments, stiffness of an arm is defined by a beam (e.g. in some embodiments, stiffness of arm 104 is defined by beam 158). In some embodiments, a beam (e.g. 158, 160) is reinforced, e.g. by thickening the arm at the region of the beam, the arm e.g. when molding the arm (e.g. from plastic). Other reinforcement methods (e.g. incorporation of a different reinforcing material e.g. metal) are envisioned and encompassed by the present invention.

In some embodiments, beams (e.g. 158, 160) provide a structural surface for the ports. In some embodiments, do not provide reinforcement and/or define the stiffness of the arm. For example, in some embodiments, a body of an arm (the body e.g. perpendicular to the beam) defines the stiffness of the arm.

In some embodiments, an arm includes one or more weaker and/or less reinforced portion, the portion of the arm designed to carry more bending and/or deformation. For example, in some embodiments, regions of arm 106 around inlets 144 bend more than other portions of arms 104, 106.

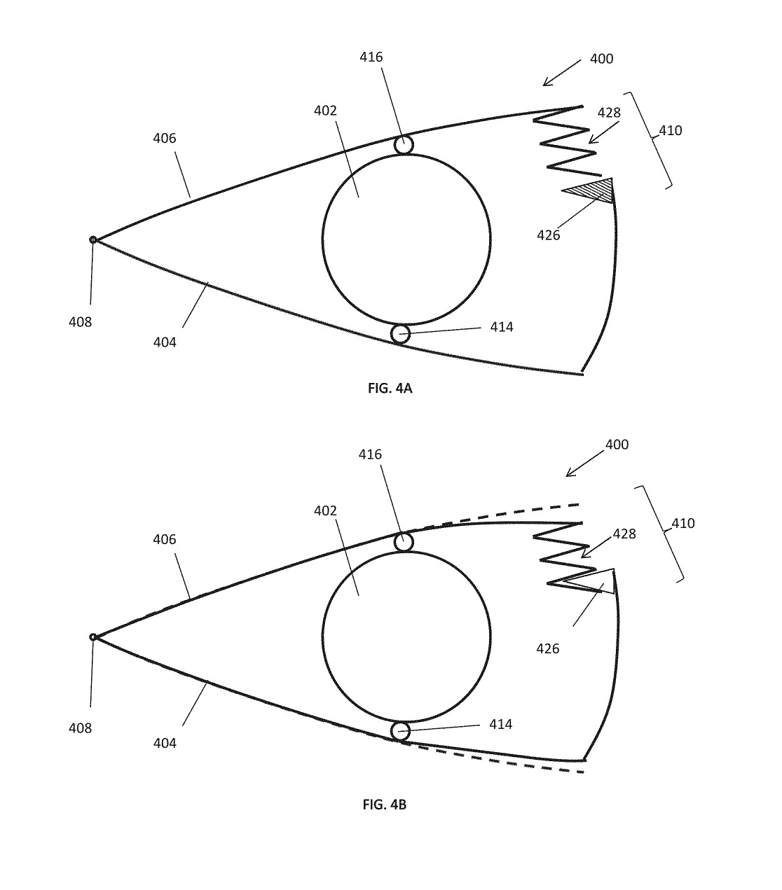

Additionally or alternatively to deformation of the arm/s, in some embodiments, grip portions elastically deform when an organizer is tightened onto a support. FIG. 5 is a simplified schematic top view of an organizer 500 tightened onto a support 502, according to some embodiments of the invention. (In FIG. 5 a cross section of a support 502 is illustrated.) Grip portions 514, 516 are elastically compressed (solid lines) when organizer 500 is tightened onto support 502. The configuration of the un-compressed, relaxed, grip portions is illustrated with dotted lines. In some embodiments, compression of grip portions (e.g. of rubber grip interfaces 118, 120 FIG. 1A, FIG. 1B) increases friction between the grip portions and the support and/or increases contact between the support and the grip portions, for example, as the shape of the grip (e.g. the grip interfaces) conforms to the shape of the support.

In some embodiments, an organizer (e.g. 400, 500) is tightened onto a support (e.g. 402, 502) by closing a ratchet mechanism. Where, for example, the ratchet mechanism includes a pawl 426, 526 and teeth 428, 528.

### Exemplary Attachment to Different Sized Supports, Exemplary Grip Portions

In some embodiments, an organizer device securely attaches to different sized supports, for example, where attachment of the organizer to the support is sufficiently secure such that forces associated with attachment and/or detachment of medical device/s to and from the organizer do not cause movement (or cause minimal movement, e.g. less that 2 mm of movement) of the organizer on the support. Where, examples of movement include; sliding of the organizer axially along the support, and/or rotating of the organizer about a support long axis.

In an exemplary embodiment, the organizer attaches to circular cross section poles (e.g. IV pole and/or bed rail) with a range of different diameters.

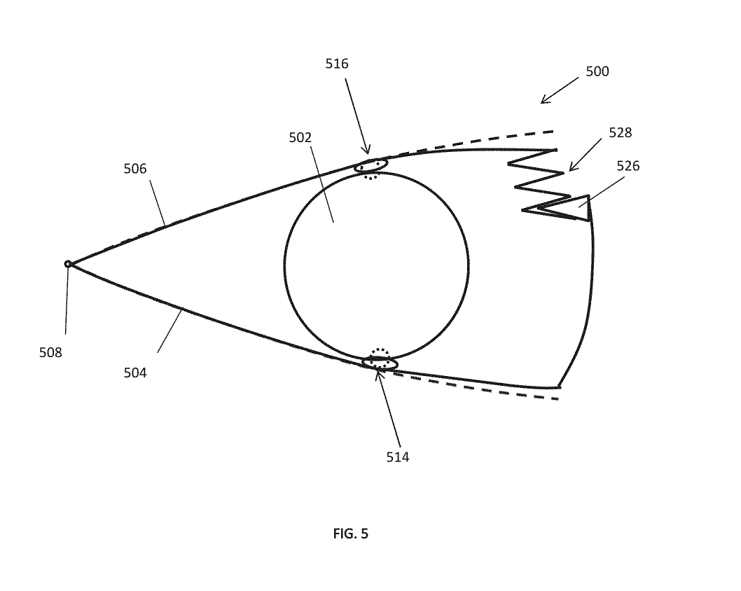

In some embodiments, the organizer attaches to rod supports with non-circular cross section, for example, poles with cross-sectional dimension which varies at different orientations. For example, in some embodiments, a rod support has oval cross section. In the case where cross-sectional dimension of a rod support is different at different orientations, the terms “maximum diameter” and “minimum diameter” refer to the dimension of the rod support cross section between the grips (e.g. grips 614, 616, FIG. 6A and FIG. 6B).

FIG. 6A is a simplified schematic top view of an exemplary organizer 600 secured onto a maximum diameter pole support 602a, according to some embodiments of the invention. FIG. 6B is a simplified schematic top view of the organizer 600 of FIG. 6A, where, in FIG. 6B, in some embodiments, the organizer is entirely closed, fitting a minimum pole support 602d, according to some embodiments of the invention. For clarity, only a cross section of supports e.g. supports 602a, 602b are illustrated.

In some embodiments, organizer 600 includes arms 604, 606 connected by a hinge 608 at proximal ends thereof and lockable by a ratchet mechanism 610 (e.g. at distal ends thereof). In some embodiments, organizer 600 includes grips 614, 616.

FIG. 6A and FIG. 6B illustrate some exemplary pole cross sections 602a, 602b, 602c, 602d, from a maximum diameter pole support 602a to a minimum diameter pole support 602d. Where, in some embodiments, the diameter of maximum diameter pole support 602a is defined by the largest lock setting on ratchet lock mechanism 610 (and, in some embodiments, the shape and/or size of the arms and/or grips). In some embodiments, a diameter of minimum diameter pole support 602d (and/or a minimum dimension of a rod support securely held between the arms) is defined by size and/or shape of the grip/s and/or arms and an extent to which grips overlap and/or interleave. In the exemplary embodiment illustrated in FIG. 6A and FIG. 6B the maximum and minimum support diameters are defined by grip portions 614, 616 and/or ratchet mechanism 610.

For example, in some embodiments, a diameter of maximum diameter support 602a is defined by the space between grip portions 614, 616 when a pawl 626 of ratchet mechanism 610 is at a first position within teeth 628 of ratchet mechanism 610 (e.g. as illustrated in FIG. 6A).

In some embodiments, a diameter of minimum diameter pole support 602d is defined by a minimum space between grip portions 616 (e.g. as illustrated in FIG. 6B). In some embodiments, grip portions are shaped to interleave, potentially facilitating smaller minimum diameters. Interleaving grip portions are described in more detail elsewhere in this document e.g. in reference to FIG.s 9A-E.

In some embodiments, the organizer is attachable to pole supports with pole diameter (of the portion providing support to the organizer) of 2-70 mm, or 10-50 mm or, lower or higher or intermediate ranges. In an exemplary embodiment, maximum pole support 602a diameter is 40 mm and minimum pole support 602d diameter is 14 mm.

### Exemplary Hold on Support, Exemplary Positioning of Grip Portions

In some embodiments, the organizer includes at least one grip pair, where both of the grips, one disposed on each arm, contacts the support when the organizer is secured to the support. In some embodiments, grips are located on rotationally different sides of the arms.

In some embodiments, the grips are positioned on the arms at the same distance from the hinge.

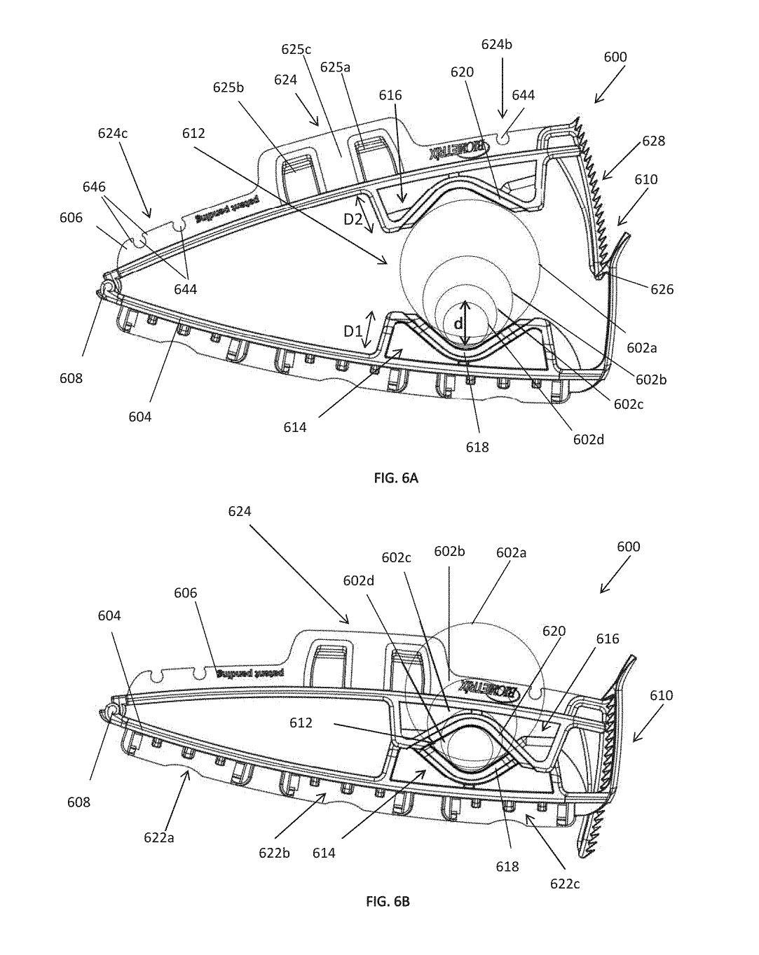

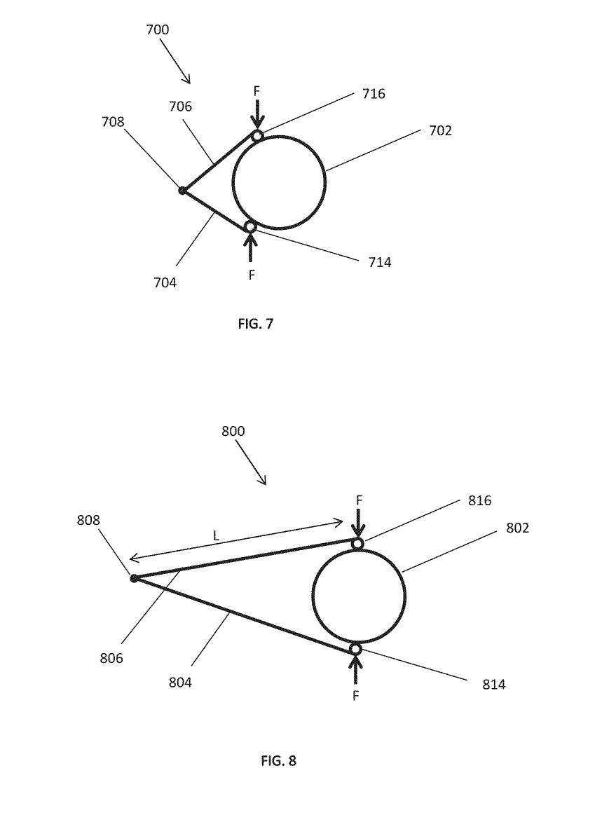

FIG. 7 is a simplified schematic top view of an organizer 700, according to some embodiments of the invention. FIG. 8 is a simplified schematic top view of an organizer 800, according to some embodiments of the invention.

The organizer of FIG. 7 shows gripping of a support 702 by the organizer 700 non-centrally. The organizer of FIG. 8 shows gripping of a support 802 by the organizer 800 centrally. In some embodiments, for example, as illustrated by FIG. 8 (contrasting with FIG. 7) arm/s of the organizer are sufficiently long and/or grip/s are positioned on the arms sufficiently distally from the hinge such that contact between the organizer and the support, even for a maximum sized support, is central on the support.

In some embodiments, an extent of the organizer, measured along an arm of the organizer from the point at which the organizer contacts the support to the hinge of the organizer, (e.g. dimension L, FIG. 8 is long with respect to a maximum diameter support. For example, 1-15 times, or 1.5-4 times, or about 2 times, or lower or higher or intermediate multiples or ranges, the diameter of the maximum diameter support.

In an exemplary embodiment, length L is about 100 mm and the maximum diameter support is about 40 mm L being 2.5 times the diameter of the maximum support.

### Exemplary Shaped Grip Portions

In some embodiments, one or more grip includes a concavity configured to accept a rod support. In some embodiments, a grip concavity is perpendicular to an axis of the hinge and in a direction towards a long axis perpendicular to the axis of the hinge of the arm on which the grip is mounted.

Referring back now to FIG. 6A, in some embodiments, one or more grip concavity includes a portion with a high curvature. In some embodiments, the radius of curvature of a high curvature portion of one or more grip corresponds with a minimum sized cross section support. For example, in some embodiments, a smallest space (perpendicular to the hinge axis) between the grip portions corresponds with a highest radius of curvature portion of a grip (e.g. the space is double the radius of curvature). In an exemplary embodiment, both grip concavities have a radius of curvature of 14 mm.

Potentially, small diameter support poles are contacted for much of their cross sectional circumference within the grip high curvature portion e.g. as illustrated in FIG. 6A by supports 602d contacting grip 614.

In some embodiments, the shape of the grip extends away from the high curvature portion, the grip shape perpendicular to an axis of the hinge is a rounded v-shape. Alternatively, in some embodiments, the grip shape perpendicular to an axis of the hinge has a sharp v-shape.

Larger diameter supports are contacted at two points by the grip, a point corresponding to each side of the v-shape e.g. as illustrated in FIG. 6A by supports 602a, 602b contacting grip 614.

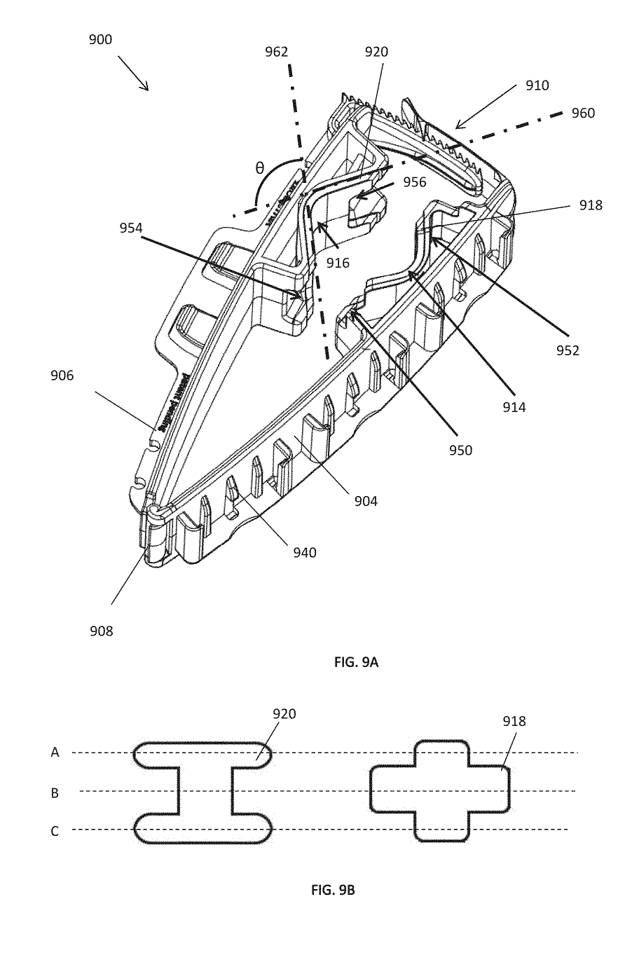

In some embodiments, one or more of said grips has a concave shape, where, in some embodiments, distal portions of the concave shape, most removed from the arm on which the grip is located are planar portions where a first axis of both said planar portions is parallel to an axis of the hinge. Referring now to FIG. 9A, in some embodiments, second axes 960 and 962 of the planar portions are orientated at θ from each other, where θ is 30-160° , or 70-110° , or approximately 90° or lower or higher or intermediate ranges or angles. In some embodiments, the second axes are symmetrical with respect to an arm long axis of the arm on which the grip is located.

In an exemplary embodiment, θ is about 90° . This angle provides strong contact for a range of sized supports (e.g. when the grips overlap on the support and/or interleave e.g. as described below). In some embodiments, θ is larger than 90° which, potentially, enables larger sized supports, however, in some embodiments, the grip is weaker.

In some embodiments, one or more grip concavity, parallel to the hinge axis, is u-shaped or semi-circle shaped. In some embodiments, compressibility of grip interfaces enables contact with different sized and/or shaped supports.

In some embodiments, the organizer contacts a sufficient proportion of a circumferential surface of pole support of different diameters. For example, in some embodiments, at least 10%, or at least 30%, or at least 50%, or at least 80% or 5-98%, or 30-98%, or lower or higher or intermediate percentages or ranges of the circumference of poles of 0.5-20 cm, or 1-10 cm, or 1-5 cm, or 1.4-4 cm diameter, or lower or higher or intermediate diameters or ranges are contacted by the organizer.

### Exemplary Overlapping And/Or Interleaving Grip Portions

In some embodiments, a minimum sized support onto which the organizer is attachable has smaller diameter than the sum of protrusion distances and/or a sum of depth of concavity of a grip pair into a space between the inner sides of the arms. For example, as illustrated in FIG. 6A and FIG. 6B where d<D1+D2 where d is the diameter of support 602d, D1 is the protrusion of grip 614 and D2 is the protrusion of grip 616.

In some embodiments, grips are shaped to interleave. FIG. 9A is a simplified schematic isometric view of an organizer 900 including grips 914, 916 shaped to interleave, according to some embodiments of the invention.

Different types of interleaving grips are envisioned and encompassed by the invention. In an exemplary embodiment, a grip 914 includes teeth 950, 952 which, as arms 904, 906 are moved towards each other, are accepted into notches 954, 956 in grip 916. In some embodiments, organizer 900 includes two grip teeth and two notches. Alternatively, in some embodiments, an organizer includes fewer or more teeth and notches, e.g. 1-10 teeth, 1-10 notches.

FIG. 9B are simplified schematic plan views of grip interfaces 918, 920 of FIG. 9A, according to some embodiments of the invention.

FIG. 9C, FIG. 9D and FIG. 9E are simplified schematic cross sections of an organizer 900, the cross section taken perpendicular to an axis of the hinge 908, according to some embodiments of the invention.

FIG. 9C illustrates a cross section of the organizer taken perpendicular to an axis of hinge 908 and along line A as marked on FIG. 9B. FIG. 9D illustrates a cross section of the organizer taken perpendicular to an axis of hinge 908 and along line B as marked on FIG. 9B. FIG. 9E illustrates a cross section of the organizer taken perpendicular to an axis of hinge 908 and along line C as marked on FIG. 9B.

FIG.s 9C-E illustrate the changing geometry of grips 914, 916 moving in an axial direction of the support 902 and/or hinge 908 and/or in a direction perpendicular to a direction of rotation of arms 904, 906 about hinge 908.

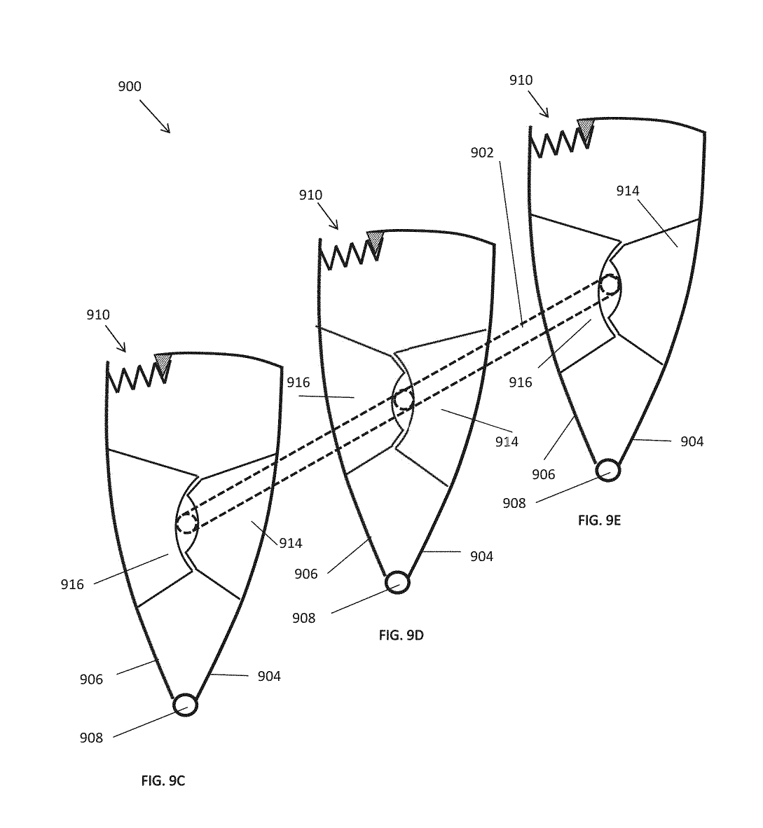

FIG. 10 illustrates interleaving grips 1014, 1016, on a support 1002, according to some embodiments of the invention. In some embodiments, a grip 1016 includes two teeth and the opposing grip 1014 includes two notches. In some embodiments, one or more grip includes both notches and teeth.

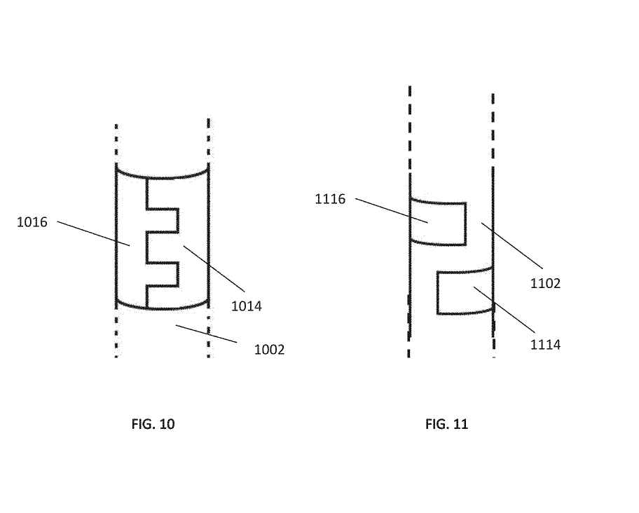

Additionally or alternatively, in some embodiments, grips overlap on a support. FIG. 11 illustrates overlapping grips 1114, 1116, on a support 1102, according to some embodiments of the invention. In some embodiments, a support 11 1114, 1116 is contacted by both grips 1114, 1116, at different axial locations on the support. In some embodiments, e.g. as illustrated in FIG. 11, grips 114, 1116 are not in close contact with (e.g. do not contact) each other.

Exemplary Organizer with a Single Concave Grip

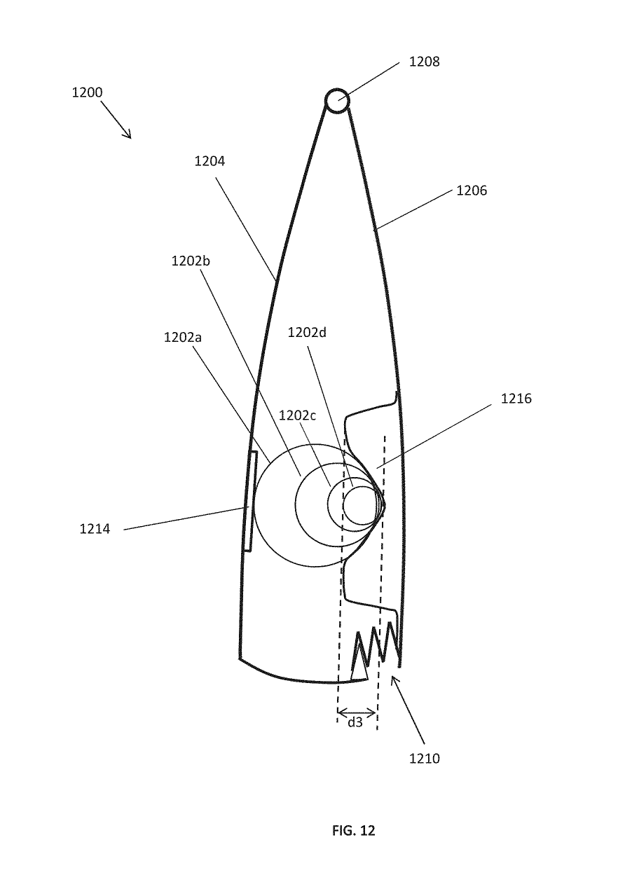

In some embodiments, an organizer device includes a single concave grip portion. FIG. 12 is a simplified schematic top view of an organizer 1200, according to some embodiments of the invention. In some embodiments, an organizer includes a single concave grip 1216 where, for example, in some embodiments, a second grip 1214 has a shape which follows a contour of the arm 1204 on which the grip is located.

A potential advantage of an organizer 1200 including a single concave grip is compatibility with a wider range of diameter supports 1202a-d. For example, with protrusion of grip structures into a space between arms 1204, 1206 being due to one grip 1216, the smallest diameter support is of diameter d3, where d3, for example is smaller than (e.g. about half of) d as illustrated in FIG. 6A.

Exemplary Organizer with More than Two Grips

In some embodiments, the organizer device includes more than one location (e.g. between organizer arms) for holding of supports. In some embodiments, different locations include different geometries, the plurality of locations potentially increasing a range of compatible supports (supports onto which the organizer is securely attached).

In some embodiments, an organizer device 1300 includes more than two grips, for example four grips, where a support is held in one of two locations between one of two grip pairs. Where a grip pair includes a grip on each arm, the grips facing each other as the arms are brought towards each other.

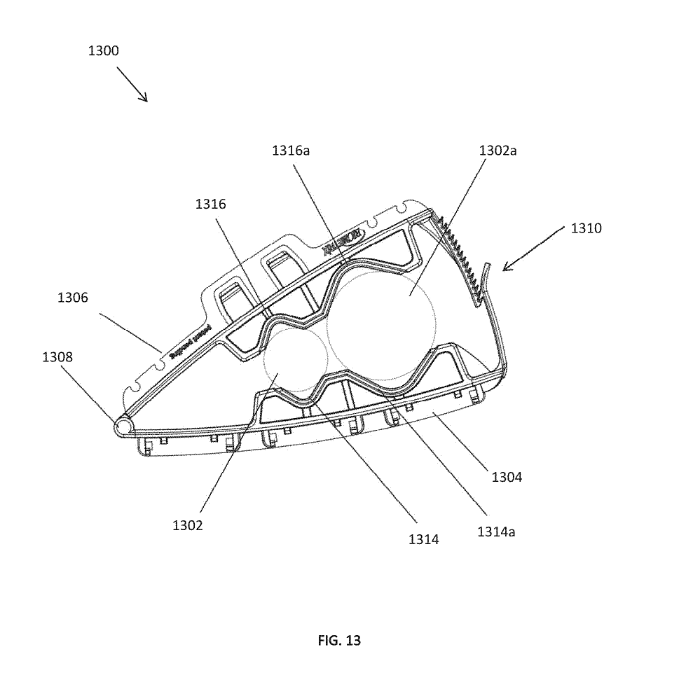

FIG. 13 is a simplified schematic top view of an organizer device 1300 including more than two grips 1314, 1314a 1316, 1316a, according to some embodiments of the invention.

In some embodiments, organizer 1300 includes a first grip pair 1314, 1316 and a second grip pair 1314a, 1316a. In some embodiments, a first grip pair and a second grip pair include different shapes, for example, the first grip pair shaped (e.g. with higher curvature) to maximize contact with smaller diameter pole supports e.g. support 1302 and the second grip pair shaped (e.g. with lower curvature) to maximize contact with larger diameter pole supports e.g. support 1302a.

In some embodiments, a first grip pair and a second grip pair have a different shape, e.g. a first grip pair shaped to maximize contact with a circular cross section support, a second grip pair shaped to maximize contact with a non-circular cross section support (e.g. as described elsewhere in this document).

In an exemplary embodiment first grip pair 1314, 1316 is sized and/or shaped for use with support poles of diameter 1.4-2.5 cm and second grip pair 1314a,1316a is sized and/or shaped for use with support poles of diameter 2-4 cm.

In some embodiments, for example, as illustrated in FIG. 13, an organizer including more than two grips does not have interleaving and/or overlapping grips.

Alternatively, in some embodiments, an organizer including more than two grips includes one or more grip pair with interleaving and/or overlapping grips.

Odd numbers of grips are envisioned and encompassed by the present invention, for example, as illustrated in FIG. 12, for example, an organizer including 3 grips, 5 grips etc.

### Exemplary Attachment of Exemplary Medical Devices

Referring back now to FIG. 1A and FIG. 1B, in some embodiments, organizer 100 includes ports (e.g. located on the arm/s) onto which medical devices are attached. In some embodiments, one or more intermediate element connects the port to a medical device. All description within this document referring to attachment of medical devices to the organizer should be understood as also referring to attachment of intermediate elements.

In some embodiments, one or more of ports 122a, 122b, 122c are slide attachment ports, where, for example, a medical device including a slide plug (or a slide plug attached to a medical device portion) is attachable a port by sliding the slide plug inside a framework of the port. In some embodiments, the framework protrudes from a surface of the arm on which the port is located (e.g. as illustrated in and FIG. 1B). Alternatively, in some embodiments, a framework for a port is recessed within a surface of the arm. In some embodiments, a port includes both recessed and protruding framework portions.

In some embodiments a slide attachment port includes a framework into which a portion of a medical device or an intermediate element attached to a medical device is slid. For example, referring to port 122a, in some embodiments, the framework is composed of two rails 136, 138 a shelf 139. In some embodiments, rails 136, 138 each have an overhanging edge.

Referring to FIG. 1A, in some embodiments, a port 122a includes one or more protrusion 166a. In some embodiments, protrusions 166a, support and/or prevent movement of a medical device attached to the port. In some embodiments, protrusions 166a maintain a space between the arm and the inserted medical device and/or intermediate element, for example, holding a secure attachment when the arms are deformed and/or relaxed (e.g. during attachment to and/or detachment from a support, e.g. during attachment and/or detachment of another medical device). In some embodiments, protrusions 166a provide support to a planar portion of a medical device element and/or intermediate element, for example, potentially improving attachment as compared to the medical device directly being supported by a curved surface of the organizer arm. For example, in some embodiments, protrusions have different dimensions, e.g. to compensate for a curve in the arm on which the protrusions are disposed. Alternatively or additionally, in some embodiments, protrusions 166a provide structural strength and/or rigidity to arm 104. In some embodiments, each slide port includes protrusions, e.g. port 122b includes protrusions 166b, port 122c includes protrusions (not indicated in FIG. 1A).

Pressure transmitter 132 (e.g. for measuring blood pressure) is an example of a medical device directly slotting into slide attachment port 122c.

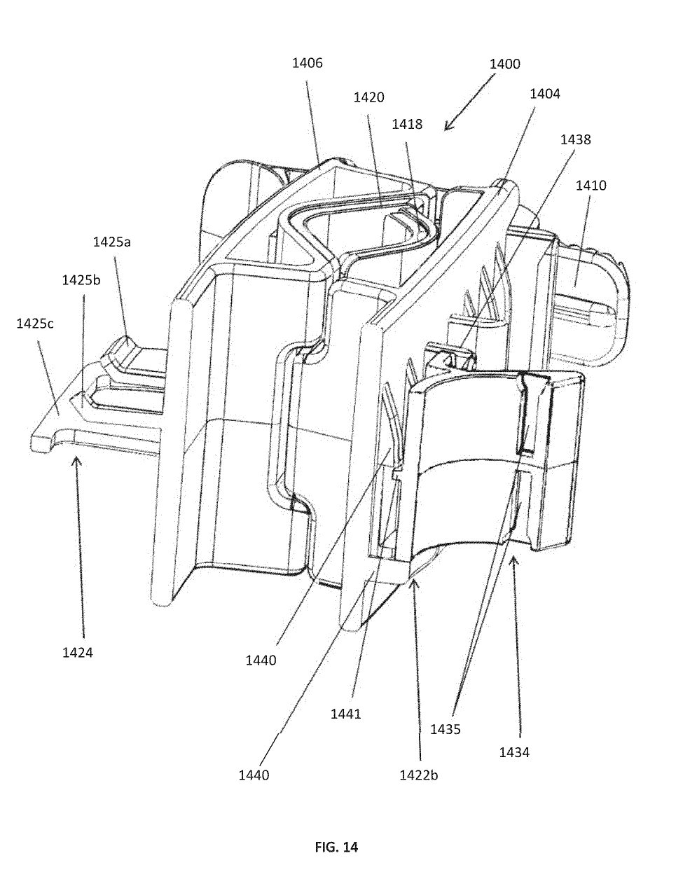

Syringe holder 134 is an example of an intermediate element between a medical device and organizer 100. In some embodiments, syringe holder 134 is used to support a syringe, e.g. as described and/or illustrated in US Patent Application No. 2014/0194778 to Uziel et al. In some embodiments, syringe holder 134 is used to support other syringe types. In some embodiments, syringe holders of different sizes are used to attach different type and/or size syringes (e.g. with different size diameter) to the organizer. For example, in some embodiments, syringe holder 1434 (referring to FIG. 14) lacks notches 1435 for attachment to a syringe. For example, in some embodiments, a syringe holder has a circular shape support for a syringe (e.g. as opposed to the semi-circular shape illustrated in FIG. 1A and FIG. 1B).

In some embodiments, a slide attachment port includes lock element/s which secure the medical device to the slide attachment socket. In some embodiments, locking element/s include a snap-fit attachment. FIG. 14 is a simplified schematic section view of a portion of an exemplary organizer, according to some embodiments of the invention. In some embodiments, FIG. 14 shows a section of the organizer illustrated in FIG. 1A and FIG. 1B, where the section is taken centrally along ports 124 and 122b, parallel to an axis of hinge 110 and perpendicular to long axes of arms 104, 106.

In some embodiments, a slide plug is inserted into the port from above, gravity, for example, holding the plug within the port. Additionally or alternatively, in some embodiments, the slide plug closely fits the port and friction prevents the plug from sliding out of the port.

Alternatively, or additionally, in some embodiments, a slide attachment port 1422b includes a snap-fit lock formed, by a stopper 1440, where syringe holder 1434 is shaped (e.g. including a protrusion 14/41) to elastically bend to slide past stopper 1440, relaxing as protrusion 1441 enters into a space below stopper 1440. In some embodiments, stopper 1440 and protrusion 1441 prevent syringe holder 1434 from sliding out of port 1422b and/or being easily removed. In some embodiments, medical device/s and/or intermediate element/s include a protrusion which, together with stopper 1440 lock the medical device and/or intermediate element in position within port 1422b. In some embodiments, more than one port includes a stopper, e.g. ports 122a and/or 122b and/or 122c. In some embodiments, a port includes more than one stopper, with medical devices and/or intermediate elements compatible with the port including one or more matching protrusion.

In some embodiments, stopper 1440 has a tapered shape, where, for example, the stopper protrudes gradually more along a direction of insertion of syringe holder 1435.

In some embodiments, a port includes one or more inlet, the inlet, for example, forming a torsional snap-fit attachment for at least portion of a medical device (and/or at least portion of an intermediate element which is attached to a medical device).

Referring to FIG. 6A, for example, in some embodiments ports 124a and 124b include inlet/s 144 for torsional snap-fit attachment where, for example, one or more edge 646 of the inlet (only indicated for one inlet in FIG. 6A) elastically bends to allow a medical device (and/or intermediate element) to enter into the inlet, the edge/s then bending back elastically to hold the medical device and/or intermediate element in position.

For example, in some embodiments, a port includes one or more cantilever which elastically deforms allowing a medical device to be positioned between the cantilever and one or more other element holding the medical device. For example, referring to FIG. 6A, in some embodiments, a port 624 includes cantilevers 625a, 625b which bend away from plate 625c, cantilevers holding a medical device onto plate 624c, for example, as illustrated in FIG. 1B where manifold 130 includes a manifold plate 130a which is held between port cantilevers 125a, 125b and plate 125c.

Other types of port and/or attachment of devices to the organizer include, for example, snap fastening (e.g. popper), snap lock, Velcro®, hook, clamp.

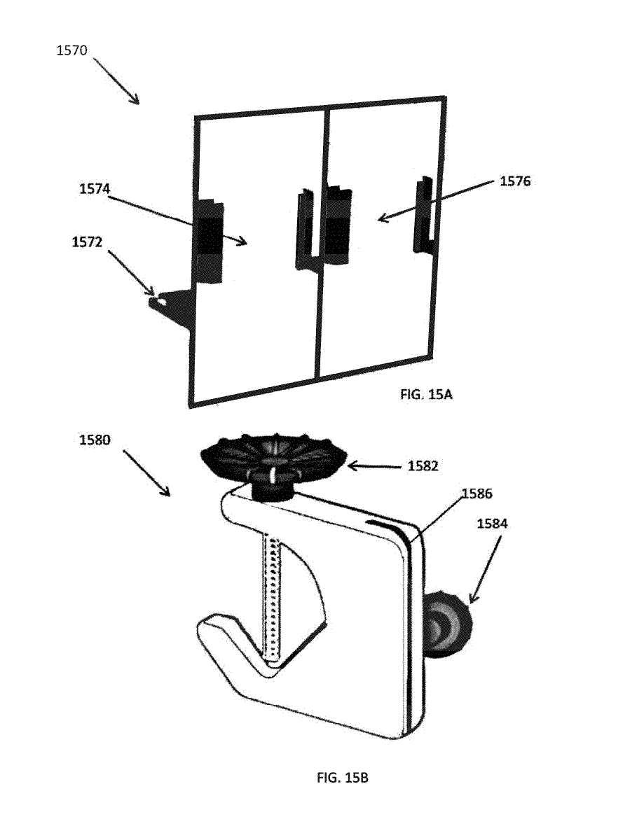

In some embodiments, ports (e.g. as described above) are located on a port plate. FIG. 15A is a simplified schematic isometric view of a port plate 1570, according to some embodiments of the invention. In some embodiments, a port plate is connected to an organizer (e.g. as described herein). For example, a port plate being an intermediate element between the organizer and medical element/s and/or additional intermediate element/s. In some embodiments, port plate 1570 is attached to a support directly, for example, by a clamp device. FIG. 15B is a simplified schematic isometric view of an attachment clamp device 1580, according to some embodiments of the invention. In some embodiments, a first screw clamp 1582 is tightened onto a support. In some embodiments, a second screw clamp 1584 is tightened onto port plate 1570, e.g. fin 1572 being inserted into slot 1586 where tightening second screw clamp 1584 reduces the size of slot 1586. Although port plate 1570 has been illustrated with two slide ports 1574, 1576, larger or smaller numbers of ports (e.g. 1, 3, 4, 5, 6, 1-10) are envisioned and encompassed by the present invention. Ports on port plate 1570 include slide port/s and/or snap fit port/s e.g. as described in this document, for example, including a slide port with a stopper.

Exemplary support

Referring back now to FIGS. 1A-B, in some embodiments, support 102 is a pole 102 e.g. an IV pole. In some embodiments, support 102 is a bed rail e.g. a hospital bed rail. In some embodiments, when the organizer is attached to a bed rail, the orientation of the organizer is that illustrated in FIG. 1A and FIG. 1B rotated by 90° (where the long axis of the sheet runs vertically).

In some embodiments, the organizer is secured to a cylindrical pole and/or cylindrical section of a pole.

In some embodiments, the organizer is attached to a portion of a rod support with non-circular cross section, for example, D shaped cross section, square cross section, oval cross section.

### Exemplary Construction of the Organizer

In some embodiments, the organizer is formed from a small number of pieces, for example, 2, or 1-4, or higher, or lower, or intermediate numbers of parts.

For example, in some embodiments, the organizer is constructed from a single plastic part defining the two arms, e.g. in some embodiments, the hinge is a living hinge.

For example, in some embodiments, the organizer is constructed from two plastic parts, one part associates with each arm, where the hinge, for example, is formed by interlocking portions of the arms.

In some embodiments, the organizer is constructed from plastic. In some embodiments, the organizer is constructed from reinforced plastic, for example, reinforced with glass fibers. In an exemplary embodiment, the organizer is constructed from glass fiber reinforced nylon.

In some embodiments, the organizer includes compressible and/or high friction portions which are affixed (e.g. by gluing) to the plastic.

In some embodiments, high friction (e.g. for grip interfaces) is provided by using high friction materials. For example, in some embodiments, grip portions include rubber and/or sponge portions e.g. natural rubber and/or silicone rubber and/or silicone sponge. For example, in some embodiments, grip interfaces (e.g. grip interfaces 118, 120 FIG. 1A) are constructed by affixing pieces of material (e.g. rubber) onto the arms which are e.g. constructed of a different material (e.g. plastic).

Alternatively or additionally, in some embodiments, high friction (e.g. for grip interfaces) is provided by texturing of the outer surface of the material, e.g. ridging. In some embodiments, friction is different in different directions, for example, in some embodiments, an organizer is designed to slide easily in one direction and remain static under force in another, for example, provided by directional texturing e.g. fins attached to a grip interface and angled in a direction non perpendicular to the surface on which they are disposed.

### General

As used herein the term “about” refers to ±20%

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

What is claimed is:

1. An organizer, configured to be mounted onto rod supports of different cross sectional sizes, where the organizer provides a hub for attachment thereto of a plurality of medical device portions, the organizer comprising:
a first arm;
a second arm;
one or more port, each port disposed on an arm, each port configured for attachment thereto of a medical device portion;
a first grip disposed on said first arm comprising a first concavity;
a second grip disposed on said second arm comprising a second concavity;
a hinge connecting said arms, where said first arm and said second arm extend distally from said hinge and are rotatable about said hinge; and
a ratchet-lock, which, when locked, connects said arms, said ratchet lock comprising:
a first ratchet-lock portion connected to said first arm;
a second ratchet-lock portion connected to said second arm;
wherein said arms are lockable by said ratchet-lock at a plurality of distances, each distance separated from adjacent distances by at least a minimum step size;
wherein, to close the organizer onto a rod support, said arms are rotated about said hinge towards each other bringing said concavities of said grips to face each other and into at least partial contact with said rod support;
wherein one or both of said arms are flexible enough in a direction perpendicular to an axis of said hinge such that total elastic deformation of the arms is at least a quarter of said minimum step size under a level of force easily applied manually.

2. The organizer of claim 1,
wherein said arms rotate about said hinge, moving in a plane perpendicular to an axis of said hinge;
wherein said first concavity comprises a first portion;
wherein said second concavity comprises a second portion;
wherein said first portion and said second portion occupy a same location perpendicular to said plane and different first and second locations in said plane when the arms are secured on rod supports with a cross sectional dimension less than a first support rod dimension.

3. The organizer of claim 2,
wherein said first concavity comprises a third portion;
wherein said third portion occupies said same location perpendicular to said plane and occupies a third region, in said plane, different to said first and second location.

4. The organizer of claim 3, wherein said third portion occupies a space between said first concavity and said second concavity perpendicular to said plane.

5. The organizer of claim 2, wherein said first concavity includes two first portions and said second concavity includes two second portions, the first portions located at different edges of said first concavity and the second portions located at different edges of said second concavity.

6. The organizer of claim 2, wherein said first support rod dimension is a sum of the depths of said first and said second concavity, a depth of a concavity measured as the largest distance perpendicular to an axis of said hinge and perpendicular to a tangential plane connecting two distal edges of the concavity.

7. The organizer of claim 1, wherein said first ratchet-lock portion is connected to a distal end of said first arm and said second ratchet-lock portion is connected to a distal end of said second arm.

8. The organizer of claim 1, wherein said first concavity comprises a first elastically compressible grip interface and said second concavity comprises a second elastically compressible grip interface.

9. The organizer according to claim 8, wherein said grip interfaces have a high friction outer surface.

10. The organizer of claim 8, wherein said arms are composed of plastic and said grip interfaces are composed of silicone rubber.

11. The organizer according to claim 1, wherein said force easily applied manually is about 30 N.

12. The organizer of claim 1, wherein said total deformation of said arms, by 1-3 mm provides an elastic force of at least 2 N.

13. The organizer of claim 1, wherein said first grip, perpendicular to said hinge axis, includes a first and a second distal end, said ends orientated between 30-160° from each other;
wherein said second grip, perpendicular to said hinge axis, includes a first and a second distal end, said ends orientated between 30-160° from each other.

14. The organizer of claim 1, wherein a trough of said first grip has a radius of curvature of about half of a minimum distance between said first grip and said second grip;
wherein a trough of said second grip has a radius of curvature of about half of a minimum distance between said first grip and said second grip.

15. The organizer of claim 14, wherein said minimum distance is defined by a final ratchet-lock locking position, where the ratchet is locked into a position where the arms are minimally separated from each other.

16. The organizer of claim 1, wherein a distance between each said grip and said hinge is at least 2 times a diameter of a support associated with a largest ratchet-lock locking position.

17. The organizer of claim 1, wherein a port on said first arm comprises a slide attachment socket.

18. The organizer of claim 17, wherein said port of said first arm comprises a snap fit lock.

19. The organizer of claim 17, wherein said slide attachment socket comprises an inlet surrounded by a framework overhanging said inlet.

20. The organizer of claim 1, wherein said first arm comprises three ports, each port comprising a slide attachment socket, each socket comprising an inlet surrounded by a framework overhanging said inlet.

21. The organizer of claim 20, wherein a port of said second arm includes a snap-fit attachment socket.

22. The organizer of claim 21, wherein said snap-fit attachment socket includes at least one arm which is elastically deflected by insertion of a portion of a medical device into the socket, said at least one arm holding said portion of a medical device in said socket.

23. The organizer of claim 1, wherein said ratchet-lock comprises:
a plurality of teeth attached to said first arm, where adjacent teeth are separated by said minimum step size; and
a pawl attached to said second arm.
